(19) United States
(12) Patent Application Publication
(10) Pub. No.: US 2019/0133450 A1
(43) Pub. Date: May 9, 2019

(54) OPTICAL FLUORESCENCE IMAGING SYSTEM AND METHODS OF USE

(71) Applicant: The Curators of the University of Missouri, Columbia, MO (US)

(72) Inventors: Raghuraman Kannan, Columbia, MO (US); Ajit Tharakan, Columbia, MO (US); Anandhi Upendran, Columbia, MO (US); Henry W. White, Columbia, MO (US)

(21) Appl. No.: 16/186,304

(22) Filed: Nov. 9, 2018

Related U.S. Application Data
(60) Provisional application No. 62/583,933, filed on Nov. 9, 2017.

(51) Int. Cl. A61B 5/00; A61B 5/02; A61M 5/00; A61B 90/00

(52) U.S. Cl. CPC A61B 5/0071; A61B 5/0077; A61B 5/4519; A61B 5/02028; A61M 5/007; A61B 2090/3904; A61B 2503/40; A61B 2560/0431; A61B 2090/3941; A61B 2090/3933; A61B 90/39

(57) ABSTRACT

An improved optical fluorescence optical imaging system is disclosed having an excitation radiation source and an emission imaging camera. The imaging system is designed for capturing video fluorescence emission images of live animal tissue in blood containing fluorescent dye. The excitation radiation source unit can easily be adapted for use with different excitation wavelengths as can the imaging camera. Hence, the system is amenable for use with a variety of different fluorescent dyes, including those with exciting wavelengths in the ultraviolet, visible, and infrared spectral regions. The small size of both the optical excitation radiation source and emission imaging camera make the entire system relatively unobtrusive to surgeons and other health care personnel in a surgical suite.

### CROSS-REFERENCE TO RELATED APPLICATIONS

This patent application claims priority to provisional U.S. patent application Ser. No. 62/583,933, filed Nov. 9, 2017 and entitled “Improved Optical Fluorescence Imaging System and Methods of Use”, the entire disclosure of which is incorporated herein by reference.

### STATEMENT REGARDING FEDERALLY SPONSORED RESEARCH OR DEVELOPMENT

Not Applicable.

### APPENDIX

Not Applicable.

### BACKGROUND OF THE INVENTION

For some time there has been interest in producing optical systems for imaging Isocyanine Green (ICG) dye injected into blood of live animals, including humans. ICG is approved by the Food and Drug Administration (FDA) of the United States of America (USA) for injection into animals, including humans.

For clarity, optical fluorescence imaging systems for use with live animals and live humans differ from those used in microscopy.

Microscopy is used primarily for capturing still images of non-living animal tissue on a slide, and features closed, light-tight constructions, and short optical path distances with high magnification.

The process of imaging tissue containing fluorescent dye in blood of a live animal, including human, by a surgical team in an operating room environment, demands longer optical path lengths. For such imaging, it is highly desirable that both the optical excitation radiation source and the emission imaging camera system be small in order to increase freedom of movement and to increase visibility by surgical team members. In addition, the imaging system requires high dynamic range and high signal-to-noise (S/N) ratio, with high rejection of noise from both fluorescent dye excitation sources and from exterior room lighting, in order to provide images with high resolution at acceptable video capture speed.

### BRIEF DESCRIPTION OF THE DRAWINGS

The accompanying drawings, which are incorporated in and form a part of the specification, illustrate the disclosed embodiments and together with the description, serve to explain certain inventive principles. In the drawings:

FIG. 1 is a schematic of the optical fluorescence imaging system of the present invention;

FIG. 2 is a schematic of the excitation radiation source of the present invention. For clarity, the housing for the components is not shown;

FIG. 3 is an artist's rendition of sixteen reflecting facets, from a total of 1152, located on the interior surface of the excitation source reflector of the excitation radiation source;

FIG. 4 is a schematic of the emission imaging camera. The laptop computer is not shown.

FIG. 5 is an image of the vein conduit in a live swine that can be visualized, located over the carotid artery below, at a time soon after initial injection of ICG dye.

FIG. 6 is an image of the vein conduit in a live swine that can be visualized, located over the carotid artery below, at a time later than initial injection of ICG dye.

FIG. 7 is an image showing perfusion of ICG dye mixed blood into surrounding tissue in a live swine. A decrease in the level of ICG dye in the carotid artery and the vein graft can be visualized.

FIG. 8 is an image showing ICG dye mixed blood entering the pulmonary artery of a live swine. The aorta is also visible.

FIG. 9 is an image showing ICG dye mixed blood leaving the pulmonary artery of a live swine. The left atrial appendage is thin walled, and therefore it is visualized as much brighter.

FIG. 10 is an image of a heart of a live swine. The valve can be seen faintly as a dark structure at the base of the pulmonary valve. The valve is dark because it has displaced ICG dye mixed blood.

FIG. 11 is an image of a heart of a live swine, as in FIG. 10 but taken at a later point in time. The valve at the base of the artery cannot be seen in this image, thereby revealing mechanical movement of the valve.

FIG. 12 is an image of a live swine that shows intestine with ICG dye mixed blood flowing through mesenteric arteries.

FIG. 13 is an image of a live swine with ICG dye mixed blood, taken at a later point in time. The image details an area of intestinal tissue and the mesenteric vessels.

FIG. 14 is an image of a live human hand with skin burn. The two left-most photographs were taken prior to injection with ICG dye. The upper left image was taken with room ambient lighting. The lower left image was taken in NIR light. The two right-most images were taken after injection of ICG dye. The two right-most images show locations of ICG mixed blood in subsurface arteries and veins; namely, the lighter colored areas, of the live human hand with skin burn.

### DETAILED DESCRIPTION OF EXAMPLE EMBODIMENTS

With reference to the above indicated figures and the following specification, an example embodiment is directed to an improved optical fluorescence optical imaging system comprising an improved excitation radiation source having small size, and an emission camera imaging system having small size, and methods.

Such example embodiments may be used with a multiplicity of fluorescent dyes, including, but not limited to ICG.

The optical fluorescence imaging system has sufficiently high dynamic range, sufficiently high S/N ratio, sufficiently small emission imaging camera, and sufficiently small optical radiation source that it can be used for capture images of tissue containing ICG in live animals, including humans, with high freedom of motion and high visibility afforded to operators and other individuals in confined spaces, such as surgeons and other health care workers in a surgical suite.

In one embodiment, the optical system for imaging is directed to use of imaging ICG fluorescence with sufficiently high dynamic range and sufficiently high S/N ratio to show blood flow in arteries and veins of live swine, and more specifically, but not limited to, perfusion of blood in transplanted veins and arteries of live swine, perfusion of blood in veins and arteries located in heart tissue of live swine, perfusion of blood in veins and arteries located in intestinal tissue of live swine, and flow of blood within a chamber of a heart sufficient to reveal motion of a leaflet on a heart valve located beneath heart muscle in live swine.

In another embodiment, the imaging system is used to obtain images from ICG dye in blood of live humans in areas with skin burn. The images showed presence of blood and blood perfusion.

Furthermore, the optical system for imaging is directed for use in diagnosis, assessment and treatment of tissue such as, but not limited to, vein and artery transplant animal tissue, mechanically damaged animal tissue, burned animal tissue, cut animal tissue, traumatized animal tissue, diseased intestinal animal tissue, heart animal tissue, and heart valve animal tissue.

In addition, the optical system for imaging is directed for use in surgical procedures for diagnosis, assessment, and treatment of animal tissue to provide increased freedom of motion and visibility for attending physicians and other attending health care personnel.

The optical system also extends to use with optical fluorescence imaging of both living animals and organisms and non-living tissue and proteins located in environments such as, but not limited to, agricultural soils, agricultural plant crops, trees, commercial slaughtering facilities, food processing facilities, general health care facilities, and human lodging facilities.

In the accordance with this disclosure, animal tissue may include human tissue, and tissue means any of the distinct types of material of which animals or plants are made, consisting of specialized cells and their products.

Also in accordance with this disclosure, the term tissue may mean one or more items such as, but not limited to, live animal tissue, non-living animal tissue, live vegetable tissue, non-living vegetable tissue, animal eggs, heart tissue, alimentary tract tissue, ureteral tissue, dermal flaps, sentinel lymph tissue, and epithelial tissue.

In areas of biomedical research and medicine, imaging of blood and blood perfusion in tissue is typically performed using an optical fluorescence dye technique wherein the dye is injected into the blood of a living animal, such as a human, and then imaging with a suitable optical camera.

On the other hand, imaging of objects such as tissue and motion of such objects is typically performed using sound based techniques, for example by generating sonograms utilizing a sound wave source and detector to image tissue features and motion.

When used alone, however, neither of these two commonly used techniques; namely, fluorescence imaging and sonograms, can provide images for both blood perfusion in tissue and information on motion of tissue, such as the motion of a valve leaflet in a beating heart.

An example embodiment discloses an improved optical fluorescence imaging system with sufficiently high dynamic range and sufficiently high S/N ratio that can be used to provide images of live animal tissue that show both blood perfusion in tissue, and motion of a valve leaflet in a beating heart of a live swine.

The size of the body of a camera of the imaging camera system of an optical fluorescence imaging system should be small is size to avoid impeding motion and visibility of those individuals utilizing the optical fluorescence imaging system.

The body of the camera in the imaging camera system may be rectangular in shape with external dimensions about 5 cm long, about 3 cm wide, and about 4 cm deep. The total volumetric size of the camera body of the imaging camera system is about 60 cm3. Of course, other camera sizes to designed to the operational constraints may be used.

To increase convenience and to decrease cost, it is desirable that an excitation radiation source has capability to be used in the ultraviolet, visible, and infrared spectral regions by changing the wavelength of the excitation source and changing the wavelength region of the excitation filter.

The excitation source has capability to be used in the ultraviolet, visible, and infrared spectral regions by changing the wavelength of the excitation source and changing the wavelength region of the excitation filter.

To increase convenience and to decrease cost, it is desirable that an excitation radiation source of an optical fluorescence imaging system has capability use light emitting diode (LED) sources, rather than use lasers, for the purpose of avoiding possible eye damage to individuals in proximity to the optical fluorescence imaging system.

The excitation source has capability to use LED sources to avoid possible eye damage to individuals in proximity to the optical fluorescence imaging system.

To increase convenience and to decrease cost, it is desirable that an excitation radiation source of an optical fluorescence imaging system has capability, without necessity to reconfigure the physical dimensions of the excitation radiation source, to use LED sources with package design types in the list including, but not limited to, tubular shaped LED, tubular shaped LED with integral lens on emission end, and flat chip.

The excitation source has capability to use LED sources with package design types such as, but not limited to, tubular shaped LED, tubular shaped LED with integral lens on emission end, and flat chip, without necessity to reconfigure the physical dimensions of the excitation radiation source.

The excitation source of the present invention has capability, without necessity to reconfigure the physical dimensions of the excitation radiation source, to add a dome-shape lens to disperse radiation emitted from LEDs having a flat chip design.

To aid in enhancing uniformity of excitation irradiance over the entire surface of tissue to be imaged, the excitation radiation source of an optical fluorescence imaging system may utilize a combination of reflector and focusing lens, rather than a lens only or a reflector only.

The excitation source may utilize a combination of both reflector and focusing lens for the purpose of enhancing uniformity of excitation irradiance over the entire surface of tissue to be imaged.

To aid in enhancing uniformity of excitation irradiance over the entire surface of tissue to be imaged, the interior surface of the reflector in an excitation radiation source of an optical fluorescence imaging system may contain a multiplicity of reflecting facets.

In the excitation source, the interior surface of the reflector located in the excitation radiation source contains a multiplicity of reflecting facets.

To increase convenience and to decrease cost, it is desirable that an excitation radiation source has capability to be used with a multiplicity of fluorescent dyes used in the ultraviolet, visible, and infrared spectral regions by changing the wavelength of the excitation source and changing the wavelength region of the excitation filter.

The excitation source may have capability to be used with a multiplicity of fluorescent dyes used in the ultraviolet, visible, and infrared spectral regions by changing the wavelength of the excitation source and changing the wavelength region of the excitation filter.

To increase convenience and to decrease cost, it is desirable that an excitation radiation source may have capability to be used in the ultraviolet, visible, and infrared spectral regions by changing the wavelength of the excitation source and changing the wavelength region of the excitation filter without a necessity to reconfigure the physical dimensions of the excitation radiation source for the purpose of allowing selection of optimal wavelengths for excitation of a multiplicity of fluorescent dyes.

The excitation source may have capability to be used in the ultraviolet, visible, and infrared spectral regions by changing the wavelength of the excitation source and changing the wavelength region of the excitation filter without a necessity to reconfigure the physical dimensions of the excitation radiation source for the purpose of allowing selection of optimal wavelengths for excitation of a multiplicity of fluorescent dyes.

To increase convenience and to decrease cost, it is desirable that an excitation radiation source may have capabilities for use with multiple fluorescent dyes by interchangeability of excitation sources and filters without necessity to reconfigure the physical dimensions of the excitation radiation source and that provides capabilities for selection of optimal wavelengths for excitation of fluorescent dye and that provides capabilities for use in an optical fluorescence imaging system in ultraviolet, visible, and infrared spectral regions.

The excitation radiation source may have capabilities for use with multiple fluorescent dyes by interchangeability of excitation sources and filters without necessity to reconfigure the physical dimensions of the excitation radiation source, that provides capabilities for selection of optimal wavelengths for excitation of fluorescent dye, and that provides capabilities for use in an optical fluorescence imaging system in ultraviolet, visible, and infrared spectral regions.

The camera in an imaging camera system of an optical fluorescence imaging system for use in a surgical suite may have high signal-to-noise (S/N) ratio to allow image capture of weak fluorescence emissions.

The camera in the imaging camera system of the present invention may have high signal-to-noise (S/N) ratio.

The camera in an imaging camera system of an optical fluorescence imaging system camera may have a dynamic range of at least about 70 decibels (dB).

The camera in the imaging camera system may also have a dynamic range of at least about 72 dB.

The camera in an imaging camera system of an optical fluorescence imaging system camera may have optical sensitivity greater than about 0.05 Lux.

The camera in the imaging camera system may have a sensitivity greater than about 0.05 Lux.

The camera in the imaging camera system of an optical fluorescence imaging system that will be used in a surgical suite can be small in size for the purpose of increasing freedom of motion and increasing visibility for attending surgeons and other health care workers.

Additionally, the size of the body of a camera in an imaging camera system of an optical fluorescence imaging system that will be used in a surgical suite can have a total volumetric size less than about 100 cm3.

The camera in the imaging camera system may also have a total volumetric size of less than about 60 cm3.

The optical excitation radiation source of an optical fluorescence imaging system that will be used in a surgical suite can be small in size for the purpose of increasing freedom of motion and increasing visibility for attending surgeons and other health care workers.

In addition, the optical excitation radiation source of an optical fluorescence imaging system that will be used in a surgical suite may have a total volumetric size less than about 600 cm3.

The optical excitation radiation source could also possess a total volumetric size of less than about 350 cm3.

A camera in an imaging camera system of an optical fluorescence imaging system for use in a surgical suite may also contain a sensor with a pixel array of at least 1500×1000 to yield sufficient resolution of tissue that is imaged.

The camera in the imaging camera system could also contain a sensor with a pixel array 1920×1200.

The camera in the imaging camera system may contain a monochrome CMOS sensor.

The camera used in the imaging camera system could also have a USB 3.0 computer interface.

The optical fluorescence imaging system may use a camera in the imaging camera system with a computer interface of the type in the list including, but not limited to, USB 3.0, USB 3.1, GiGE, and Camera Link.

The camera of the imaging camera system may have a small size, have high dynamic range, high sensitivity, and have high image resolution.

The imaging lens selected for the imaging camera body may be cylindrical in shape. The imaging lens extends about 3.2 cm from the front point of the camera body when it is mounted in the camera body, and at its largest point the imaging lens is about 3.6 cm diameter.

Other lens are commercially available for use with the imaging camera body that are suitable in size, wherein such lens have small size, and may be useful to select desired size for field-of-view (FOV), for optical zoom features, and for remote control over adjustable lens operating parameters.

A lens hood is attached to the distal end of the imaging lens. The size of the lens hood may have an interior depth of about 2.0 cm, and circular interior opening about 3.2 cm in diameter at its distal end (i.e., its optical acceptance opening), and an interior opening about 2.3 cm in diameter. The exterior shape of the imaging lens hood may be uniformly cylindrical over its entire length, and is about 3.5 cm in diameter.

Including camera body, lens, and imaging lens hood, the overall length of the imaging camera system is about 11.2 cm.

The lens may have a 25 focal length, and f-stop is adjustable from f1.4 to f16 in steps of ½ of an f-stop.

Dimensions of the imaging lens hood provide capability to exclude incoming incident optical rays for which the angle of incidence measured with respect to a normal to the emission filter surface exceeded 41 degrees.

Lack of maintaining sufficiently high S/N ratio throughout all components of a fluorescence optical imaging system will degrade image quality.

Use of light emitting diodes (LEDs), rather than use of one or more lasers or laser diodes (LDs) of equivalent radiation intensity, as an optical excitation radiation source eliminates speckle arising from the coherent radiation from lasers, including LDs, as, wherein such laser speckle can also become an annoyance and distraction to users of a fluorescence optical imaging system for imaging ICG fluorescent dye in tissue of a live animal. Such annoyance and distraction can reduce performance by attending health care personnel. Furthermore, use of lasers, including LDs, as an excitation source endangers operators of a fluorescence optical imaging system by generation of coherent radiation that can cause eye damage.

Such embodiments utilize LEDs as an excitation radiation source to improve operator performance and to increase operator safety.

When radiation from an optical source is incident on a short pass filter designed to eliminate longer wavelengths in an optical system to obtain fluorescence images of subsurface blood of animal tissue containing ICG dye, it is preferable to use a reflector with steep sides, i.e., a conical-shaped reflector with interior sides forming small angles with respect to the cone axis, rather than with shallow sides, for the purpose of minimizing the fraction of light incident on the filter be incident at an oblique angle, and thereby have an increased likelihood of passing through the filter. Such design will increase the S/N ratio of the optical system.

When radiation from an optical source is incident on a short pass or band pass excitation radiation filter designed to eliminate longer wavelengths in an optical system used to obtain fluorescence images of subsurface blood of animal tissue containing ICG dye, it is preferable to use a reflector with steep sides, i.e., a conical-shaped reflector with interior sides forming angles in the range 14 degrees to 22 degrees, and more preferentially about 18 degrees with respect to the cone axis, rather than with more shallow sides, for the purpose of minimizing the fraction of light incident on the filter be incident at an oblique angle, and thereby have an increased likelihood of passing through the filter. Incorporation of such design in an excitation source as is done in the present invention will increase the S/N ratio of the optical system by decreasing light from the excitation source from being introduced directly into the emission band to be imaged.

The optical fluorescence imaging system may have a conical-shaped reflector with interior sides forming an angle 18 degrees with respect to the cone axis.

To improve S/N ratio for an optical fluorescence imaging system it is important to exclude from the fluorescence band to be imaged those wavelengths that may exist in the excitation source.

Prior art does not teach that that the S/N ratio and safety of a fluorescence optical imaging system are improved by use of an excitation radiation source comprising a multiplicity of LEDs as source of radiation in combination with a reflector for said LED excitation radiation source that has a conically shaped reflecting interior that preferentially directs said excitation radiation onto either a short pass or band pass excitation filter at incident angles less than 45 degrees from a normal to said excitation filter surface, and wherein said excitation filter is characterized as having an optical density value at least 3.5 (i.e., OD3.5), and more preferably an optical density about OD4, for rays incident at incident angles less than 25° from a normal to the excitation filter surface.

The optical fluorescence imaging system may have an excitation filter with OD4.

Prior art does not teach that that S/N ratio of an ICG optical imaging system is improved by use of an excitation radiation source comprising a multiplicity of LEDs as source of radiation in combination with a reflector for said LED excitation radiation source that has a conically shaped reflecting interior bearing a multiplicity of reflecting facets, wherein reflecting facets preferentially direct said excitation radiation onto a more uniform illumination pattern at the surface of tissue to be imaged.

Use of both a focusing lens and a reflector in the excitation radiation source provides more uniform illumination on the surface of tissue to be imaged than would use of only a lens or only a reflector.

In some embodiments, the excitation source can be utilized without use of a lens.

In some embodiments, the excitation source can be utilized without use of a reflector.

In some embodiments, the excitation source can be utilized without use of a reflector that contains a multiplicity of reflecting facets located on the interior surface of the reflector.

Failure to employ proper methods in use of a fluorescence optical imaging system will degrade image quality.

High S/N ratio is a desired characteristic of an optical fluorescence imaging system. The use of methods that reduce the S/N ratio of the optical fluorescence imaging system are to be avoided. The optical excitation radiation necessary for optical fluorescence imaging is itself a source of noise. An excess of optical excitation radiation at surface of tissue to be imaged lowers the S/N ratio for an optical fluorescence imaging system.

The methods disclosed herein use the minimal amount of excitation radiation at surface of tissue to be imaged necessary to obtain images that show high contrast of fluorescence emission.

Historically, currently used methods for obtaining optical fluorescence images teach use of a high level of excitation radiation intensity to the surface of tissue to be imaged, and thereby teach away from the disclosed methods.

Photonic radiation is itself a source of noise, and therefore use of excitation radiation intensity values that are higher than necessary to obtain sufficient image quality will cause a reduction of the overall S/N ratio of a fluorescence optical imaging system.

In a review, Alander et al. (A Review of Indocyanine Green Fluorescent Imaging in Surgery, Int'l J. of Biomedical Imaging, Vol. 2012, Article ID 940585, 26 pages) report recent ICG fluorescent imaging applications, technology, instrumentation, and methods. The review describes several important sources of noise that can degrade S/N ratio of an optical system to obtain fluorescence images of blood of animal tissue containing ICG dye.

In Table 9, col. 1, page 9, Alander et al. provide an example of excitation light radiation intensities and attenuation factors at various points for use of optical system to obtain fluorescence images of blood of animal tissue containing ICG dye. In this example, the value for excitation radiation intensity at the surface of animal tissue was calculated to be 80 W/m2 (equivalent to 8 mW/cm2).

Neither Alander et al. nor references contained therein state that excitation radiation intensity per se is a source of noise in a fluorescence optical imaging system optical system for imaging Indocyanine green (ICG) fluorescent in animal tissue, nor did Alander et al., nor did references contained therein.

An optical fluorescence imaging system should have sufficiently high dynamic range and sufficiently high S/N ratio that images of blood perfusion and mechanical motion of subsurface tissue of a live animal containing ICG dye can be imaged when the fluence rate on the surface of tissue being imaged is less than about 6 mW/cm2.

The optical fluorescence imaging system may have sufficiently high dynamic range and sufficiently high S/N ratio such that blood perfusion and mechanical motion of subsurface tissue of a live animal containing ICG dye can be imaged when the fluence rate on the surface of tissue being imaged is less than about 6 mW/cm2.

An optical fluorescence imaging system should have sufficiently high dynamic range and sufficiently high S/N ratio such that blood perfusion and mechanical motion of subsurface tissue of a live animal containing ICG dye can be imaged when the fluence rate on the surface of tissue being imaged is less than about 3 mW/cm².

The optical fluorescence imaging system may have sufficiently high dynamic range and sufficiently high S/N ratio such that blood perfusion and mechanical motion of subsurface tissue of a live animal containing ICG dye can be imaged when the fluence rate on the surface of tissue being imaged is less than about 3 mW/cm².

An optical fluorescence imaging system may also have sufficiently high dynamic range and sufficiently high S/N ratio such that blood perfusion and mechanical motion of subsurface tissue of a live animal containing ICG dye can be imaged when the fluence rate on the surface of tissue being imaged is less than about 1 mW/cm2.

In other embodiments, the optical fluorescence imaging system may have sufficiently high dynamic range and sufficiently high S/N ratio that images of blood perfusion and mechanical motion of subsurface tissue of a live animal containing ICG dye can be imaged when the fluence rate on the surface of tissue being imaged is less than about 1 mW/cm2.

Susan L. Troyan et al. (The FLARE™ Interoperative Near-Infrared Fluorescence Imaging System: A First-in-Human Clinical Trial in Breast Cancer Sentinel Lymph Node Mapping, Ann. Surg. Oncol. 2009 Oct. 16(10): 2943-2956; page 6) state that their FLARE™ imaging system designed for ICG fluorescence guided surgery utilized a fluence rate of 14 mW/cm². Fluence rate is equivalent to irradiance.

Optical fluorescence imaging systems described in prior art are deficient due to characteristics including, but not limited to, camera that is large and thereby restricts freedom of motion and restricts visibility of attending surgeon or other critical health care workers or operators, lack of camera with high dynamic range, lack of high S/N ratio in each and all optical components, emission band to be imaged is contaminated with long wavelength radiation from the excitation radiation source that has leaked through short pass excitation filter or imaging band pass filter, a camera with low image resolution, and S/N ratio of the optical fluorescence imaging system insufficient to obtain images of blood perfusion and mechanical motion of subsurface tissue when the fluence rate on the surface of tissue being imaged is less than about 4 mW/cm2.

The emission imaging camera system may utilize either one or of a multiplicity of individual emission imaging camera system units. If more than one such emission imaging camera is utilized to capture images of the tissue of interest, then different and complementary image views can be attained. A multiplicity of image views can be utilized to increase surface areas images for irregular surfaces, and provide a sense of depth perception. 3-D images can also be attained with proper image capture and display.

The optical excitation radiation source may utilize either one or of a multiplicity of individual optical excitation radiation source units. If more than one such optical excitation radiation source units is utilized to radiate the surface of tissue to be imaged, then higher power per cm2 can be attained.

The optical excitation radiation source can be utilized to provide a radiation intensity of about 10 mW/cm2 at the surface of tissue to be imaged.

The optical fluorescence imaging system can be utilized to obtain high quality images of ICG mixed blood in tissue when the radiation intensity at the surface of the tissue is about 10 mW/cm2.

The optical excitation radiation source can be utilized to provide a radiation intensity of about 20 mW/cm2 at the surface of tissue to be imaged.

The optical fluorescence imaging system can be utilized to obtain high quality images of ICG mixed blood in tissue when the radiation intensity at the surface of the tissue is about 20 mW/cm2.

The optical excitation radiation source can be utilized to provide a radiation intensity of about 40 mW/cm2 at the surface of tissue to be imaged.

The optical fluorescence imaging system can be utilized to obtain high quality images of ICG mixed blood in tissue when the radiation intensity at the surface of the tissue is about 40 mW/cm2.

The optical excitation radiation source can be utilized to provide a radiation intensity of about 80 mW/cm2 at the surface of tissue to be imaged.

The optical fluorescence imaging system can be utilized to obtain high quality images of ICG mixed blood in tissue when the radiation intensity at the surface of the tissue is about 80 mW/cm2.

The optical excitation radiation source can be utilized to provide a radiation intensity of about 200 mW/cm2 at the surface of tissue to be imaged.

The optical fluorescence imaging system can be utilized to obtain high quality images of ICG mixed blood in tissue when the radiation intensity at the surface of the tissue is about 200 mW/cm2.

The optical excitation radiation source can be utilized to provide a radiation intensity between about 200 mW/cm2 and about 400 200 mW/cm2 at the surface of tissue to be imaged.

The optical fluorescence imaging system can be utilized to obtain high quality images of ICG mixed blood in tissue when the radiation intensity at the surface of the tissue is between about 200 mW/cm2 and about 400 mW/cm2.

Fluorescence optical imaging systems described in prior art are deficient in methods employed to obtain high quality images due to one or more steps in the list comprising, but not limited to, use of excessive excitation radiation intensity at surface of tissue to be imaged, and use of inhomogeneous intensity of excitation radiation on tissue to be imaged.

The optical fluorescence imaging system can be re-configured for use with a multiplicity of fluorescent dyes by substitution of commercially available components for the commercially LED excitation radiation source, short pass excitation filter, and band pass emission filter without changes in mechanical and optical construction of the optical fluorescent imaging system.

The optical fluorescence imaging system can be used with fluorescent dyes that emit in the infrared, visible, and ultraviolet regions of the optical spectrum.

The optical fluorescence imaging system can be used to image items in the list including, but not limited to, tissue, proteins, peptides, antibodies, blood, serum, lymph fluid, and plant sap.

The optical fluorescence imaging system can be used to obtain three-dimensional (3-D) images of items in in the list including, but not limited to, tissue, proteins, peptides, antibodies, blood, serum, lymph fluid, and plant sap.

The optical fluorescence imaging system can be used to obtain images for use in visualization of molecular processes or structures.

The optical fluorescence imaging system can be used to obtain images using fluorescent dyes.

The term fluorescent dyes as recited throughout this disclosure encompasses fluorescent dyes including, but not limited to, Indocyanine Green (ICG), Indo-1, Ca saturated, Indo-1, Ca2+, Cascade Blue BSA pH 7.0, Cascade Blue, Alexa 405, LysoSensor Blue pH 5.0, LysoSensor Blue, DyLight 405, DyLight 350, BFP (Blue Fluorescent Protein), Alexa 350, 7-Amino-54-methylcoumarin pH 7.0, Amino Coumarin, AMCA conjugate, Coumarin, 7-Hydroxy-4-methylcoumarin, 7-Hydroxy-4-methylcoumarin pH 9.0, Difluoro-7-Hydroxy-4-methylcoumarin pH 9.0, Hoechst 33342, Pacific Blue, Hoechst 33258, Hoechst 33258-DNA, Pacific Blue antibody conjugate pH 8.0, PO-PRO-1, PO-PRO-1-DNA, POPO-1, POPO-1-DNA, DAPI-DNA, DAPA, Marina Blue, SYTOX Blue-DNA, CFP (Cyan Fluorescent Protein), eCFP (Enhanced Cyan Fluorescent Protein), 1-Anilinonaphthalene-8-sulfonic acid (1,8-ANS), Indo-1, Ca free, 1m8-ANS (1-Anilinonaphthalene-8-sulfonci acid), BO-PRO-1-DNA, BOPRO-1, BOBO-1-DNA, SYTO 45-DNA, evoglow-Bs1, evoglow-Bs2, Auramine 0, DiO, LysoSensor Green pH 5.0, Cy 2, LysoSensor Green, Fura-2 Ca2+ sup>, SYTO 113-DNA, YO-PRO-1-DNA, YOYO-1-DNA, eGFP (Enhanced Green Fluorescent Protein), LysoTracker Green, GFP (S65T), [BODIPY FL, MeOH], Sapphire, BODIPY FL conjugate, [MitoTracker Green FM, MeOH], Fluorescein 0.1 M NaOH, Calcein pH 9.0, Fluorescein pH 9.0, Calcein, [Fura-2, no Ca], Fluo-4, FDA, DTAF, Fluorescein, Fluorescein antibody conjugate pH 8.0, CFDA, FITC, Alexa Fluor 488 hydrazide-water, DyLight 488, 5-FAM pH 9.0, FITC antibody conjugate pH 8.0, Alexa 488, Rhodamine 110, Rhodamine 110 pH 7.0, Acridine Orange, Alexa Fluor 488 antibody conjugate pH 8.0, BCECF pH 5.5, PicoGreenDNA quantitation reagent, SYBR Green I, Rhodaminen Green pH 7.0, CyQUANT GR-DNA, NeuroTrace 500/525, green fluorescent Nissl stain-RNA, DansylCadaverine, Rhodol Green antibody conjugate pH 8.0, Fluoro-Emerald, Nissl, Fluorescein dextran pH 7.0, Rhodamine Green, 5-(and-6)-Carboxy-2′-, 7′-dichlorofluorescein pH 9.0, [DansylCadaverine, MeOH], eYFP (Enhanced Yellow Fluorescent Protein), Oregon Green 488, Oregon Green 488 antibody conjugate pH 8.0, Fluo-3, BCECF pH 9.0, SBFI-Na+, Fluo-3 Ca2+, [Rhodamine 123, MeOH], FlAsH, Calcium Green-1 Ca2+, Magnesium Green, DM-NERF pH 4.0, Calcium Green, Citrine, LysoSensor Yellow pH 9.0, TO-PRO-1-DNA, Magnesium Green Mg2+, Sodium Green Na+, TOTO-1-DNA, Oregon Green 514, Oregon Green 514 antibody conjugate pH 8.0, NBD-X, DM-NERF pH 7.0, [NBD-X, MeOH], CI-NERF pH 6.0, Alexa 430, Alexa Fluor 430 antibody conjugate pH 7.2, CI-NERF pH 2.5, [Lucifer Yellow, CH], LysoSensor Yellow pH 3.0, [6-TET, SE pH 9.0], Eosin antibody conjugate pH 8.0, Eosin, 6-Carboxyrhodamine 6G pH 7.0, [6-Carboxyrhodamine 6G, hydrochloride], Bodipy R6G SE, [BODIPY R6G, MeOH], 6 JOE, Cascade Yellow antibody conjugate pH 8.0, Cascade Yellow, mBanana, Alexa Fluor 532 antibody conjugate pH 7.2, Alexa 532, Erythrosin-5-isothiocynate pH 9.0, [6-HEX, SE pH 9.0], mOrange, mHoneydew, Cy 3, Rhodamine B, Dil, 5-TAM RA-MeOH, Alexa 555, Alexa Fluor 555 antibody conjugate pH 7.2, DyLight 549, [BODIPY TMR-X, SE], [BODIPY TMR-X, MeOH], PO-PRO-3-DNA, PO-PRO-3, Rhodamine, Bodipy TMR-X conjugate, POPO-3, Alexa 546, BODIPY TMR-X antibody conjugate pH 7.2, Calcium Orange Ca2+, TRITC, Calcium Orange, Rhodaminephalloidin pH 7.0, MitoTracker Orange, [MitoTracker Orange, MeOH], Phycoerythrin, Magnesium Orange, R-Phycoerythrin pH 7.5, 5-TAMRA, Rhod-2, FM 1-43, Rhod-2 Ca2+, Tetramethylrhodamine antibody conjugate pH, FM 1-43 lipid, LOLO-1-DNA, dTomato, DsRED, Dapoxyl (2-aminoethyl) sulfonamide, Tetramethylrhodamine dextran pH 7.0, Fluor-Ruby, Resorufin pH 9.0, mTangerine, LysoTracker Red, Lissaminerhodamine, Cy 3.5, Rhodamine Red-X antibody conjugate pH 8.0, [Sulforhodamine 101, EtOH], JC-1 pH 8.2, JC-1, mStrawberry, MitoTracker Red, [MitoTracker Red, MeOH], X-Rhod-1 Ca2+, Alexa Fluor 568 antibody conjugate pH 7.2, Alexa 568, 5-ROX pH 7.0, 5-ROX (5-Carboxy-X-rhodamine, triethylammonium salt), BO-PRO-3-DNA, BOPRO-3, BOBO-3-DNA, Ethidium Bromide, ReAsh, Calcium Crimson, Calcium Crimson Ca2+, mRFP, mCherry, Texas Red-X antibody conjugate pH 7.2, HcRed, DyLight 594, Ethidium homodimer-1-DNA, Ethidiumhomodimer, Propidium Iodide, SYPRO Ruby, Propidium Iodide-DNA, Alexa 594, [BODIPY TR-X, SE], [BODIPY TR-X, MeOH], BODIPY TR-X phallacidin pH 7.0, Alexa Fluor 610 R-phycoerythrin streptavidin pH 7.2, YO-PRO-3-DNA, Di-8-ANEPPS-lipid, YOYO-3-DNA, Nile Red-lipid, Nile Red, DyLight 633, mPlum, TO-PRO-3-DNA, DDAP pH 9.0, [Fura Red, high Ca],Allophycocyanin pH 7.5, APC (allophycocyanin), [Nile Blue, EtOH], TOTO-3-DNA, Cy 5, [BODIPY 650/665-X, MeOH], Alexa Fluor 647 R-phycoerythrin streptavidin pH 7.2], DyLight 649, Alexa 647 Fluor 647 antibody conjugate pH 7.2, Alexa 647, Fura Red Ca2+, Atto, 647, [Fura Red, low Ca], Carboxynaphthofluorescein pH 10.0, Alexa 660, Alexa Fluor 660 antibody conjugate pH 7.2, Cy 5.5, Alexa Fluor 680 antibody conjugate pH 7.2, Alexa 680, Alex Fluor 700 antibody conjugate pH 7.2, Alexa 700, [FM 4-64, 2% CHAPS], and FM 4-64.

The excitation radiation source can be re-configured for use with a multiplicity of fluorescent dyes by substitution of commercially available components for the commercially LED excitation radiation source, short pass excitation filter, and band pass emission filter without requiring major changes in mechanical and optical construction of the optical fluorescent imaging system.

The excitation radiation source can be used with fluorescent dyes that emit in the infrared, visible, and ultraviolet regions of the optical spectrum.

The excitation radiation source can be used to image items in the list including, but not limited to, tissue, proteins, peptides, antibodies, blood, serum, lymph fluid, and plant sap.

By suitable addition of a three-dimensional (3-D) image unit to the imaging camera system, with such 3-D units available commercially from a multiplicity of commercial vendors, the optical fluorescence imaging system may contain an optical radiation excitation source having proper wavelength output for the fluorescent dye being employed that can be used to obtain 3-D images of items in in the list including, but not limited to, tissue, proteins, peptides, antibodies, blood, serum, lymph fluid, and plant sap.

The excitation radiation source can be used to obtain images for use in visualization of molecular processes or structures.

The excitation radiation source can be used to obtain images using a multiplicity of fluorescent dyes.

Many fluorescent dyes can be utilized by making modifications to the excitation radiation source, wherein such required modifications would be either minor or incidental in nature.

Many fluorescent dyes can be utilized by making modifications to the excitation radiation source, wherein such required modifications would be incorporated into a general design that would allow such changes to be either minor or incidental in nature.

Optical fluorescence optical imaging systems described in prior art are deficient in mechanical and optical construction in combination with small size that allows re-configuration from use of a particular fluorescent dye by means that do not require any change in mechanical construction of the disclosed optical fluorescent imaging system.

As one example use, the optical fluorescence imaging system can be re-configured from use with ICG fluorescent dye to use of Cy5 fluorescent dye by making the following changes using commercially available components, with each type of component being available from the same respective vendor, and with same mechanical dimensions.

| COMPONENT | ICG Dye | | Cy5 Dye | |
|---|---|---|---|---|
| LED radiation source | 770 | nm | 625 | nm (a.) |
| Short pass excitation filter | 775 | nm | 650 | nm (b.) |
| Single band emission filter | 832 +/− 18.5 | nm | 692 +/− 20 | nm (c.) |

(a.) Vendor and Stock No.: Roithner Lasertechnik, Stock No. LED625-03d.
Note:
23 mW
(b.) Vendor and Stock No.: Edmund Optics, Stock No. 84-725
(c.) Vendor and Stock No.: Semrock, Stock No. FF01-692/40-25

Furthermore, none of these component changes require any changes in mechanical and optical construction of the optical fluorescent imaging system. The values for wavelength for the Cy5 components are representative. As noted in (a.), the power on the 625 nm LED is 23 mW, rather than 18 mW for the 770 nm LED, however both are specified for 50 mA DC current. Many fluorescent dyes can be utilized by either none or only minor modifications to the optical fluorescence imaging system.

Use of the optical fluorescence imaging system can be re-configured from use with ICG fluorescent dyes other than Cy5 fluorescent dye by making changes similar in nature to those made for Cy5 dye using commercially available components, with each type of component being available from either the same or a different optical parts vendor, and with same or sufficiently equal mechanical dimensions such that replacement can be made without mechanical alteration of the optical fluorescence imaging system.

Optical fluorescence optical imaging systems described in the prior art are deficient in mechanical and optical construction in combination with small size that allows re-configuration from use of a particular fluorescent dye by means that do not require any change in mechanical construction of the optical fluorescent imaging system.

The review by Alander et al. does not teach that the volume of the body of an imaging camera system of optical system for imaging ICG fluorescent dye should be small in order to improve freedom of movement by surgeons and other attending health care individuals.

The review by Alander et al. also does not teach that the volume of an optical radiation source in an optical fluorescent imaging system should be small in order to improve visibility freedom of movement by surgeons and attending health care individuals.

Furthermore, the S/N ratio of an optical system to obtain images of fluorescence dye in tissue can be increased by limiting the light entering the imaging camera by placement of a cylindrically shaped hood that extends from the distal end of the camera lens towards the emission source, wherein the hood helps eliminate light with wavelengths outside the emission band of interest since stray light of longer wavelength, e.g., from fluorescent room lights, with wavelengths outside the band of interest and are incident at large angles from the normal may pass through the filter. The hood thereby optimizes the fraction of total light incident on the emission filter light to that the fraction of light emitted by the exciting source be incident that is normal to the surface of the excitation filter. The interior of such hood should have a surface topology that enhances light absorption, and a coating such as black paint that is highly light absorbing.

Also, the S/N ratio of an optical system to obtain images of tissue containing ICG fluorescent dye can be increased by increasing the uniformity of intensity of the excitation radiation that is incident on the tissue to be imaged. Furthermore, the S/N ratio of an optical system to obtain images of tissue containing a fluorescent dye can be increased by increasing the uniformity of intensity of the excitation radiation that is incident on the tissue to be imaged.

The uniformity of intensity of excitation radiation can be increased by use of both an optical reflector and an optical focusing lens in combination, rather than by use of a lens only, or by use of a reflector only. Hence, use of both an optical reflector and an optical focusing lens increases S/N ratio of an optical system to obtain fluorescence images of blood in tissue containing a fluorescent dye.

The disclosed system contains both an optical reflector and an optical focusing lens.

The uniformity of intensity of excitation radiation can be increased by use of a multiplicity of reflecting facets on the interior of a reflector, rather than only use of reflector with a smooth, highly reflecting surface without facets. The use of a properly faceted reflector surface helps diffuse light reflected from said surface, and helps in avoiding brighter and darker contrasting areas on a tissue surface to be illuminated for obtaining images by use of ICG fluorescent dye optical imaging system. Use of a properly faceted reflector surface further increases image quality of an optical system that is used to obtain fluorescence images of tissue containing ICG fluorescent dye.

Furthermore, the uniformity of intensity of excitation radiation can be increased by use of a multiplicity of optical reflecting facets on the interior of an optical reflector. The use of a properly faceted optical reflector surface helps diffuse light reflected from said optical reflector surface, and helps in avoiding brighter and darker contrasting areas on a tissue surface to be illuminated for obtaining images by use of a fluorescent dye optical imaging system. Use of a properly faceted optical reflector surface further increases image quality of an optical system that is used to obtain fluorescence images of tissue containing a fluorescent dye.

Example embodiments contain an optical reflector with a multiplicity of optical reflecting facets on the interior of the optical reflector.

Hence, a need still exists for an improved optical imaging system for monitoring blood perfusion in tissue of a live animal using ICG fluorescent dye. Furthermore, a need still exists for an improved optical fluorescence imaging system having sufficiently high dynamic range and sufficiently high S/N ratio for uses including, but not limited to, monitoring perfusion of fluids, subsurface fluid perfusion, protein, tissue, and subsurface motion of tissue using one or more fluorescent dyes.

Furthermore, a need still exists for an improved excitation radiation source of small size for use with optical fluorescent imaging system ratio for uses including, but not limited to, monitoring perfusion of fluids, subsurface fluid perfusion, protein, tissue, and subsurface motion of tissue using one or more fluorescent dyes.

Even furthermore, a need still exists for an improved optical imaging system of small size for monitoring blood perfusion in tissue of a live animal using ICG fluorescent dye. Furthermore, a need still exists for an improved optical fluorescence imaging system having sufficiently high dynamic range and sufficiently high S/N ratio for uses including, but not limited to, monitoring perfusion of fluids, subsurface fluid perfusion, protein, tissue, and subsurface motion of tissue using one or more fluorescent dyes, wherein the size of the imaging camera is sufficiently small to allow increased freedom of movement and visibility for surgeons and other attending health care individuals in a surgical suite.

Furthermore, a need still exists for an improved excitation radiation source for use with optical fluorescent imaging system ratio for uses including, but not limited to, monitoring perfusion of fluids, subsurface fluid perfusion, protein, tissue, and subsurface motion of tissue using one or more fluorescent dyes, wherein the size of the optical excitation radiation source is sufficiently small to allow increased freedom of movement and visibility for surgeons and other attending health care individuals in a surgical suite.

Furthermore, a need still exists for an improved imaging camera system for use with optical fluorescent imaging system ratio for uses including, but not limited to, monitoring perfusion of fluids, subsurface fluid perfusion, protein, tissue, and subsurface motion of tissue using one or more fluorescent dyes, wherein the size of the optical excitation radiation source is sufficiently small to allow increased freedom of movement and visibility for surgeons and other attending health care individuals in a surgical suite.

Among the objects of the disclosure, therefore, are the provisions of an improved optical system used obtain fluorescence images of tissue containing fluorescent dye, and the provision of methods thereof.

Briefly, therefore, the present invention is directed to an excitation radiation source that contains a multiplicity of LEDs, and wherein each LED emits principally at wavelengths in the range for exciting a fluorescent dye injected in tissue to be imaged, with such LED emission occurring principally in the forward direction into the more narrow end of a conically-shaped optical reflector truncated at both ends, and wherein the optical reflector has walls that form an acute angle with respect to the conical axis, and wherein the optical reflector has a multiplicity of optically reflecting facets, with all such optically reflecting facets preferentially being uniformly distributed over the interior surface, and with such optically reflecting facets protruding slightly into the interior of the conically shaped optical reflector a distance about one percent of the average diameter of the conically-shaped optical reflector, and wherein each of the lateral dimensions of an optically reflecting facet are about 1 mm×1 mm in lateral dimension, and about 1 mm high, and wherein a short pass excitation filter having diameter of about or greater that the exit diameter of the conically-shaped optical reflector, and for which the transition wavelength from short pass to high blocking is located about a wavelength mid-point between the longest exciting wavelength for the fluorescent dye and the lowest emission wavelength of the fluorescent dye, and wherein the differences in optical density between the two wavelength regions of the short pass excitation filter is at least about OD4, (it provides attenuation of 10,000 to the longer wavelength region) for excitation radiation incident on the excitation filter surface within an incidence angle within about 45 degrees of a surface perpendicular, and an optical lens having diameter of about or greater that the exit diameter of the conically-shaped optical reflector, and such optical lens having focal length and distance from the emission LEDs to form an illuminated area on tissue to be imaged.

The optical system is further directed to a imaging camera with multi-pixel array sensor with high dynamic ranges and high quantum efficiency (QE) in the wavelength region of the fluorescent emission band to be captured, wherein the imaging camera preferentially has an interface for computer control, and wherein the imaging camera is fitted with a collection lens, and wherein collection lens is fitted with an emission band pass filter having a rejection ratio for wavelengths outside the pass band of about OD6, and with band pass region center in the emission region of fluorescent dye having high intensity, and wherein lens is also fitted with a hood that extends outward from the outer surface of the filter. The interior surface of the hood contains circular grooves and is coated with a black paint for the purposes of absorbing stray light incident on the interior surface of the hood, such that incoming stray light that is oriented more than about 20 o with respect to the normal of the lens would not be incident on the emission band pass filter.

The imaging camera is small in size, and furthermore the imaging camera having a suitable matching lens allows the imaging camera to be located a distance from the tissue to be imaged that is sufficiently long that placement allows ample freedom of movement and visibility for personnel who are either working with or observing the tissue. Such personnel may include, but is not limited to, surgeons, health care workers, and scientists.

The optical system is further directed to an optical fluorescence imaging system having an optical excitation radiation source having small size and an imaging camera system having small size and high dynamic range, wherein the optical fluorescence imaging system has sufficiently high S/N ratio for obtaining images of tissue containing one or more fluorescent dyes, wherein such images show perfusion of fluids, perfusion of subsurface fluid, protein, tissue, and subsurface motion of tissue.

The disclosed method involves the steps of sedation of a live animal to be imaged, expose the tissue to be imaged, inject fluorescent dye into blood stream of the animal, wait until fluorescent dye enters tissue to be imaged, adjust excitation radiation intensity at the surface of tissue to be imaged to a value sufficient to obtain images with high resolution and high contrast, and for which any desired video images can be obtained at an adequate frame rate, and/or acceptable still shot exposure time.

Multiple excitation radiation source units may be employed to achieve a desired excitation radiation intensity at tissue surface to be imaged. For convenience to the user of the optical fluorescent imaging system, it is suggested that the imaging camera may be placed directly above the tissue surface, i.e., normal to the tissue surface, and the excitation source about 45 degrees to the side, and also closer to the tissue surface. Placement of the excitation radiation source, and also the camera, as may be required for convenience to users of the optical fluorescence imaging system, such as surgeons in a surgery suite.

Adjustment of the illumination intensity from an excitation radiation source unit may be made by employing a pulsed source of DC current to the LEDs.

For the purpose of reducing system noise that arises due to the inherent noise associated with a photon source, the lowest excitation radiation intensity should be used that provides high contrast images of fluorescence emission from the tissue of interest, thereby contributing to an overall increase in the S/N ratio for the fluorescent dye optical imaging system.

For a live animal, including a live human, the method for obtaining improved optical fluorescence images of surface and subsurface blood in tissue of a live animal containing ICG dye with high S/N ratio is comprised of the following steps:
Anesthetize the animal,
Expose the organ to be imaged,
Illuminate the tissue to be imaged using excitation radiation source,
Focus the imaging camera system on the tissue to be imaged,
Inject ICG dye into blood stream of animal,
Wait until fluorescent dye enters tissue to be imaged, and
Obtain video and/or still images of the tissue of interest using the lowest excitation intensity commensurate with obtaining high image resolution, high contrast, and acceptable video frame rate and/or still shot exposure time.

For tissue, the method for obtaining improved optical fluorescence images of fluorescent dye in tissue with high S/N ratio is comprised of the following steps:
Expose the tissue to be imaged,
Illuminate the tissue to be imaged using excitation radiation source,
Focus the imaging camera system on the tissue to be imaged,
Inject fluorescent dye into tissue,
Wait until fluorescent dye perfuses tissue to be imaged, and
Obtain video and/or still images of the tissue of interest using the lowest excitation intensity commensurate with obtaining high image resolution, high contrast, and acceptable video frame rate and/or still shot exposure time.

The invention is further directed to a monochrome imaging camera with CMOS sensor with dynamic range of about 72 dB, with about 30% quantum efficiency (QE) in the wavelength region around 832 nm (i.e., in the fluorescent emission band captured), with a 1920 pixels×1200 pixels array, and wherein the imaging camera has a USB 3.0 interface for input/output and control, and is fitted with an f1.4-f16 lens, and wherein the lens is fitted with a 25 mm diameter, OD6 band pass filter having a 37 nm pass window centered at 832 nm, and wherein the lens is also fitted with a hood that extends outward from the outer surface of the filter. The interior surface of the hood contains circular grooves and is coated with a black paint for the purposes of absorbing stray light incident on the interior surface of the hood such that incoming stray light that is oriented more than about 41 degrees with respect to the normal of the lens will be excluded from being incident on the exterior surface of the emission band pass filter. During operation and image collection on live swine, the camera unit was placed directly above the animal, while the excitation source was placed at an angle about 45 degrees from the vertical. The separation distance between excitation light source and surface of live animal tissue to be imaged was about 30 cm, and the separation distance between the camera unit and the surface to be imaged was about 70 cm.

Even furthermore, embodiments of the disclosure are directed to an optical fluorescence imaging system, wherein the optical excitation radiation source is small, and battery powered, and wherein the imaging camera system is battery powered.

The optical system is further directed to a CCD color imaging camera mounted beside the CMOS monochrome camera for use when it is desirable to record RGB color images of tissue surface.

The optical system is further directed to use of either a battery or an electronic DC power supply to provide current to the LEDs.

In accordance with the disclosure, it has been discovered that an improved optical fluorescence imaging system for monitoring blood perfusion and mechanical action in animal tissue using a fluorescent dye can be fabricated. Surprisingly, the improved optical imaging system has sufficiently high dynamic range and sufficiently high S/N ratio that it is capable for monitoring blood perfusion and mechanical motion of animal tissue using ICG fluorescent dye.

Also, in accordance with the disclosure, an improved optical radiation excitation source has been discovered that is useful for optical fluorescence imaging and can be used in an optical fluorescence imaging system for monitoring blood perfusion and mechanical action in animal tissue using a fluorescent dye can be fabricated.

Furthermore, the small size of the imaging camera body make the optical fluorescence imaging system useful for use in a surgical suite.

Even furthermore, the small size of the optical excitation radiation source make the optical fluorescence imaging system useful for use in a surgical suite.

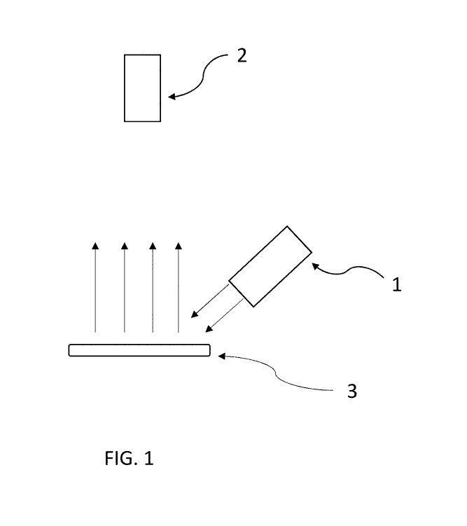

Referring to the accompanying drawings in which like reference numbers indicate like elements, FIG. 1 illustrates a schematic diagram of an improved optical fluorescence imaging system of the present invention for monitoring blood perfusion and mechanical action in animal tissue using a fluorescent dye. ICG and other fluorescent dyes may be utilized. Such a system is one that can be used to image blood perfusion and mechanical action in live animal tissue.

Other sources for excitation radiation delivered to the surface of tissue to be illuminated to excite blood containing ICG include LDs. A battery is used to provide a constant DC current through the LEDs. Other sources for providing power to the LEDs include a constant current power supply. Other cameras may include cameras with cooled imaging sensors to further reduce thermal background noise. Other cameras may include cameras with CMOS imaging sensors, sensors cooled below ambient (room) temperature, sensors that feature dual readout amplifiers on each pixel to further reduce readout noise.

Other methods for measuring blood perfusion in live animal tissue include cine radiography. Other methods for measuring position and movement of subsurface animal tissue include sonograms.

Again referring to FIG. 1, there is shown an excitation radiation source.

The excitation radiation source 1 is capable of directing a beam of excitation radiation to the surface of tissue to be imaged.

Again referring to FIG. 1, there is shown a schematic of an emission imaging camera 2 for capturing emission radiation from fluorescent dye in tissue to be imaged. The schematic is not to scale.

Again referring to FIG. 1, there is shown tissue 3 to be imaged using the optical fluorescence imaging system of the present invention.

The optical system of the present invention was used to obtain fluorescence images of tissue of a live, sedated swine containing ICG dye injected into a vein.

In a particularly embodiment, the optical fluorescence imaging system was used to image carotid artery grafting of a live swine. The fluorescent dye used was ICG.

The optical fluorescence imaging system may have an excitation radiation source and an emission imaging camera.

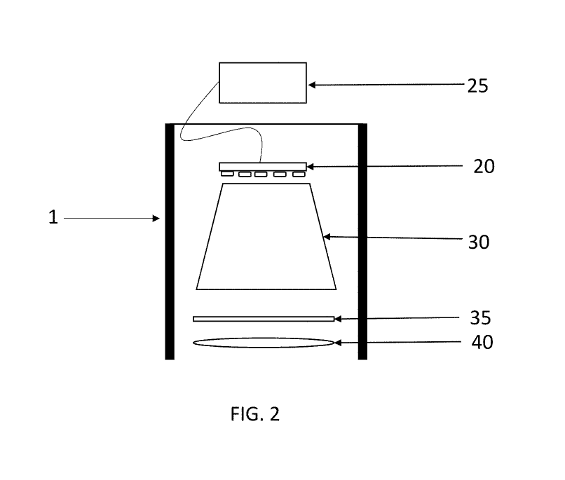

FIG. 2 is a schematic of the excitation radiation source 1. For clarity, some mechanical details of the housing for the components are not shown. The schematic is not to scale.

The excitation radiation source contains an assembly of LEDs 20. In one embodiment, the excitation radiation source contains an assembly of seven LEDs. In another embodiment, the excitation radiation source contains an assembly of twenty three LEDs. The positions of the LEDs in the excitation radiation source were spaced approximately uniformly within a circle. Each LED in the excitation radiation source emits about 18 mW of power at a central wavelength of about 770 nm, with peak power emitted in a cone in the forward direction from the emitter end with such cone having a spread of about +/−15° from the forward direction. The total number of LEDs can be varied.

The number of LEDs in the excitation radiation source may be increased to more than seven to provide increased optical radiation. As an example only, the number of LEDs may be increased to ten, or more.

Total power emitted from the excitation source was measured using a SCIENTECH Model Astral AA30.

The excitation radiation source contains a 12 V DC battery 25. If seven LEDs are in a series circuit, then a small 12 V DC lead-acid battery was utilized to supply current through each LED, with an added resistor in series with the seven LEDs to avoid excessively large current. The total series resistance was adjusted to provide a current of about 50 mA, or less, through each LED.

The 12 V DC battery was small, and therefore portable.

The excitation radiation source contains a cone-shaped excitation source reflector 30. LED radiation is emitted into the narrow end of the interior of the cone-shaped reflector. The interior surface of the excitation source reflector was oriented about 18° from the central axis of the cone-shaped reflector. The interior surface of the excitation source reflector contained about 1152 reflecting facets that were distributed uniformly over its interior surface.

The excitation radiation source contains a 775 nm short pass excitation filter 35. The short pass filter is 50 mm diameter, with an optical rejection factor of at least about OD4 for wavelengths located above 775 nm, for the purpose of eliminating wavelengths that might have otherwise appeared in the ICG emission band to be imaged, thereby reducing the S/N ratio of the optical fluorescence imaging system.

The LED radiation was then focused by a double convex lens 40.

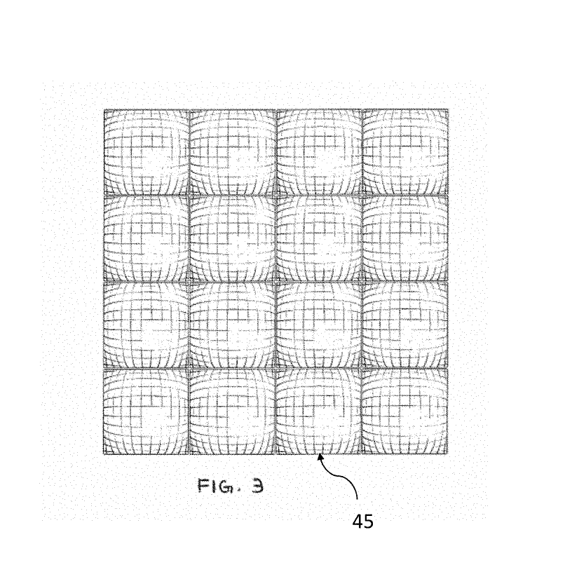

A schematic illustrating sixteen optically reflecting facets, located on the interior surface of the excitation source reflector of the excitation radiation source, is shown in FIG. 3.

A representative optically reflecting facet 45 is identified. The total number of facets on the interior surface of the excitation source is about 1152. All 1152 facets are not shown.

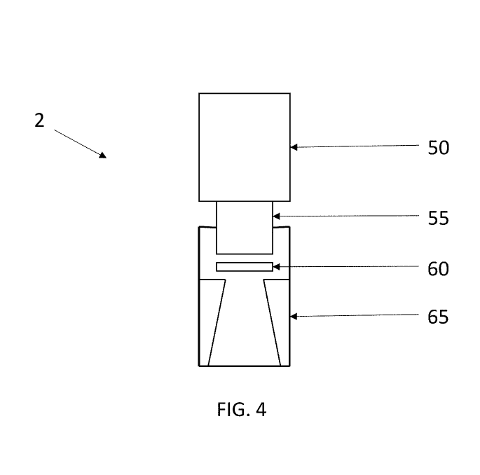

A schematic of the emission imaging camera is shown in FIG. 4. The schematic is not to scale.

The emission imaging camera 2 contains a monochrome camera body 50. The monochrome camera body contained a CMOS sensor having a quantum efficiency of at least about 30%. The monochrome camera recorded still and video images of ICG mixed blood in swine animal tissue. The CMOS sensor contained an array of 1920×1200 pixels. The size of each pixel was 5.86 microns×5.86 microns. The dynamic range of the imaging sensor is about 73 dB. The sensor was operated at room temperature, without cooling. Video capture rate used for image collection was about 30 frames/sec (fps).

The emission imaging camera contained a lens 55 that was attached to the camera body of the imaging camera for focusing the image onto the imaging sensor located within the emission imaging camera. The lens had an adjustable f-number over the range f-1.4 to f-16.

The emission imaging camera contains an emission band pass filter 60. The emission band pass filter has a diameter of about 25 mm. The emission band pass filter has an optical density of at least about OD6 for radiation outside a band of width about 37 nm band, centered at about 832 nm. The emission band pass filter is located at the entrance to the lens of the emission imaging camera. Light cannot enter the emission imaging camera without passing through the emission band pass filter.

The emission imaging camera contained a lens hood 65 mounted on the lens at its entrance. The lens hood accepted radiation with incidence 41° or less, as measured from the central optic axis of the lens.

The emission imaging camera system was controlled and provided power by connection between its USB 3.0 port to the USB 3.0 port of a laptop computer (laptop computer is not shown in FIG. 4), with imaging software provided by the camera vendor via internet download from vendor's website. The imaging software was used to download images from the imaging camera system.

The laptop could operate from power supplied by internal batteries. As such, the laptop was a portable device.

The laptop could also operate from power supplied by a 115 V AC, 60 Hz source. Other power sources may also be used.

The optical fluorescence imaging system is small in size and is also is highly portable since batteries can be used to provide power to the optical excitation radiation source, the imaging camera system, and the laptop used for camera power and control.

The high portability of the optical fluorescence imaging system allows it to be used in remote locations, such as those locations in the list including, but not limited to, remote emergency medical locations, military field hospital locations, agricultural field locations, and remote locations in need of water quality assessment.

The small size and flexibility of positioning of both the optical excitation radiation source and the emission imaging camera system of the optical fluorescence imaging system allows it to be used to obtain images of objects without necessity to move such objects. Such objects may include, but are not limited to, plants in an agricultural field, an injured individual in a remote location, polluted water reservoirs, determination of analytes in solution, and water quality control units.

An additional color camera (not shown) was used also to obtain RGB color images of tissue area imaged. The dimensions of the body of the color camera were the same as those of the body of the monochrome imaging camera, with a total volume of about 60 cm3. The color camera obtained power from the laptop used for the emission imaging camera system, and is therefore portable.

For a live animal, including a live human, the general method for obtaining improved optical fluorescence images of surface and subsurface blood in tissue of a live animal containing ICG dye with high S/N ratio is comprised of the following steps:
1. Anesthetize the animal,
2. Expose the organ to be imaged,
3. Illuminate the tissue to be imaged using excitation radiation source,
4. Focus the imaging camera system on the tissue to be imaged,
5. Inject ICG dye into blood stream of animal,
6. Wait until fluorescent dye enters tissue to be imaged, and
7. Obtain video and/or still images of the tissue of interest using the lowest excitation intensity commensurate with obtaining high image resolution, high contrast, and acceptable video frame rate and/or still shot exposure time.

If the animal is human, then prior to such trial, the maximum allowed amount of ICG that can safely be injected into a human should be determined. One goal of a human trial would be to optimize the ICG concentration for use in humans.

In a particularly preferred embodiment of the present invention the fluorescent dye is ICG and the tissue is swine tissue. In this particularly embodiment, the optical fluorescence imaging system utilized one excitation radiation source unit that contained seven LEDs. This excitation radiation source provided an irradiance less than or about 1 mW/cm² at the surface of live swine tissue being imaged.

### FIRST EXAMPLE

Two different concentrations of stock solutions of ICG were prepared; namely, 1 mg/ml and 2.5 mg /ml. Preparation of the stock solutions were made in accordance with descriptions provided by Oh et al. (Intraoperative combined color and fluorescent images-based . . . ; J. Thoracic and Cardiovascular Surgery, 146, 6, 2013) for use in sentinel lymph node mapping.

The left and right carotid were grafted using a jugular vein. Grafting success was visualized using the improved optical system of the present invention, and subsequently validated using coronary angiography.

The monochrome camera was mounted on a stand and was located 100 cm directly above the field-of-view (FOV) of the surface of the swine tissue to be imaged. The camera lens aperture was set to f1.4.
1. The excitation source was mounted on the same stand, and located 30 cm from the surface of the swine tissue to be imaged, at an angle 30° from the line between camera and field-of-view (FOV).
2. To visualize the left carotid artery grafting, 3 ml of the 1 milligram/ml ICG stock solution, corresponding to 70 microgram/kg for swine, was injected intravenously through an auricular catheter, and followed by a flush of 8 ml sterile saline solution.
3. Upon injection of ICG, ICG fluorescence was observed via monochrome images in both the native and grafted vein, confirming blood flow through the veins.
4. The injection of ICG lead to clear visualization of vasculature structures, including veins.
5. When the graft was occluded with vascular clamps on the bypass graft, then blood flow became obstructed as revealed by the monochrome camera ICG images. The blood flow was observed in the native vessel upon occlusion, as expected, and further confirmed by the ICG fluorescence images.
6. The staining and the ICG fluorescence persisted for more than 15 minutes.
7. After the ICG fluorescence images of the left side carotid images were recorded and stored, the right side carotid grafting images were recorded.
8. For right side carotid imaging, a decision was made to reduce the concentration of

ICG in the swine, re-locate the monochrome camera to be closer to the surface of tissue to be imaged (still directly above the tissue to be imaged), and to reduce the size of camera lens aperture in order to obtain higher resolution ICG images of the right side carotid graft.
9. 1.5 ml of the 1 milligram/ml stock solution, corresponding to 35 microgram/kg of swine, was injected intravenously through the auricular catheter, and followed by an 8 ml flush of sterile saline solution.
10. Upon injection of the ICG solution, flow of blood containing ICG was observed by use of the monochrome camera; however, flow was not continuous. Fluorescence images of ICG blood recorded by the monochrome camera revealed that flow was obstructed by a blood clot located in the bypass graft. However, following application of gentle massage to the clotted region by the attending surgeon, the ICG fluorescence showed blood flow to become continuous and normal, thereby suggesting that blood flow resumed upon removal of the clot.
11. The ICG fluorescence images obtain using the method wherein the camera lens aperture was reduced to f8, and concentration of ICG in swine blood was also reduced. The resulting images were sharp; however, some details associated with fine structures were absent, and graininess of recorded images increased.
12. Following ICG imaging of the left and right carotid, then coronary angiography was performed. Results from coronary angiography and ICG imaging were consistent.

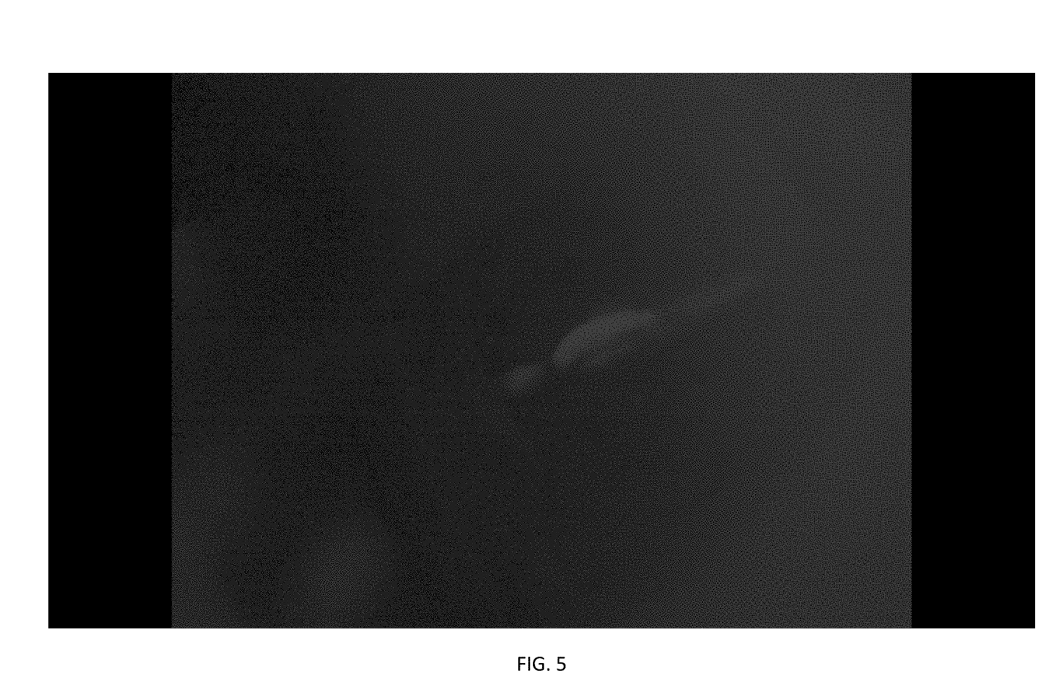

FIG. 5 is an image of the vein conduit in a live swine that can be visualized, located over the carotid artery below, at a time soon after initial injection of ICG dye.

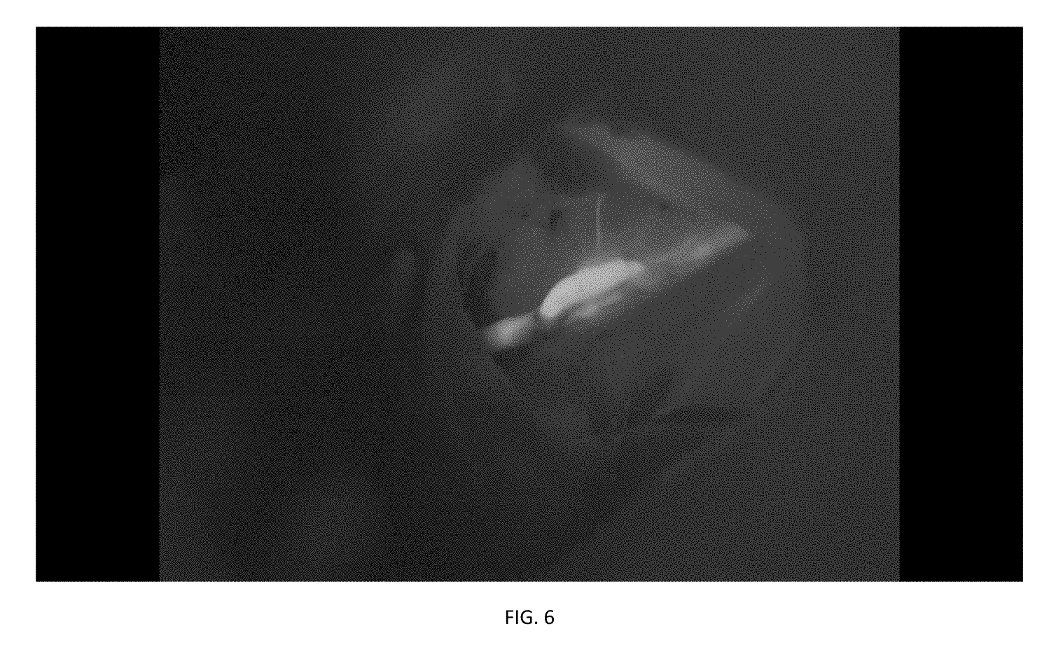

FIG. 6 is an image of the vein conduit in a live swine that can be visualized, located over the carotid artery below, at a time later than initial injection of ICG dye.

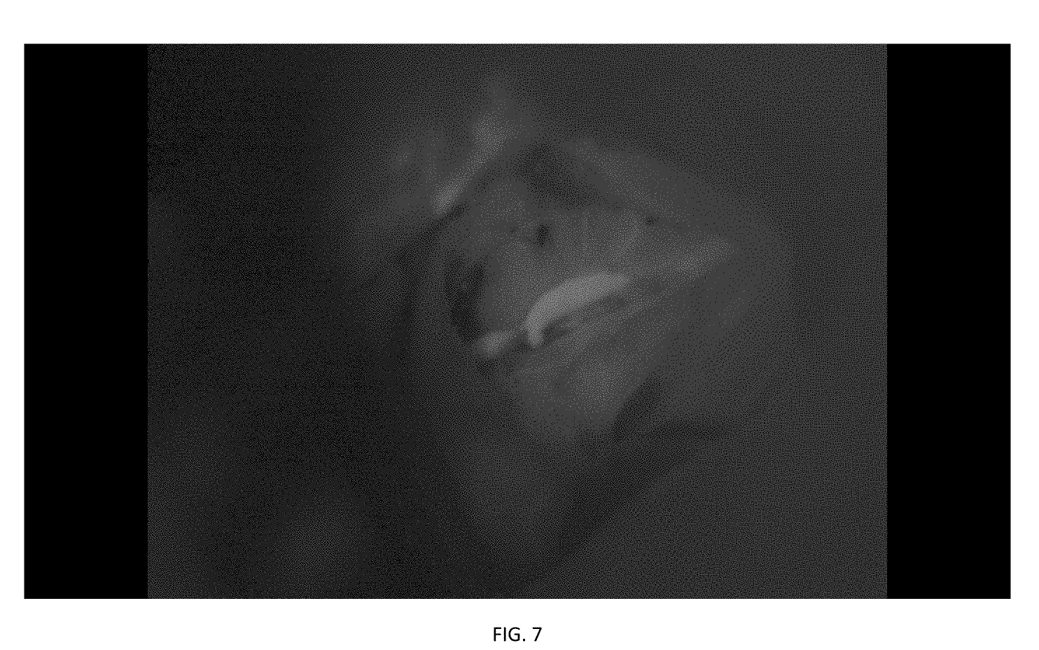

FIG. 7 is an image showing perfusion of ICG dye mixed blood into surrounding tissue in a live swine. A decrease in the level of ICG dye in the carotid artery and the vein graft can be visualized.

### SECOND EXAMPLE

Immediately following the first example, the optical imaging system was used to image blood in pulmonary valves inside the pulmonary artery of a live swine.

Visualization of the pulmonary valves inside the pulmonary artery of swine were made using the lower concentration of ICG. 3 ml of the 1 milligram/ml ICG stock solution was injected intravenously through the auricular catheter.

Blood perfusion and location was monitored by recording images of ICG fluorescence using the monochrome camera. Imaging of the pulmonary valves was partially successful. Blood flow through one of the pulmonary valves was monitored successfully, while imaging of the two others were difficult. Fine structures were still visible.

Therefore, to visualize the second and third valves, it was decided to increase the concentration of ICG utilized in the injection.

Then, 3 ml of the 2.5 milligram/ml stock solution was injected by IV through the auricular catheter. The pulmonary artery was then monitored. The resolution increased; however, in order to visualize deeper structures it was concluded that increased sensitivity of the optical camera system needed to be increased.

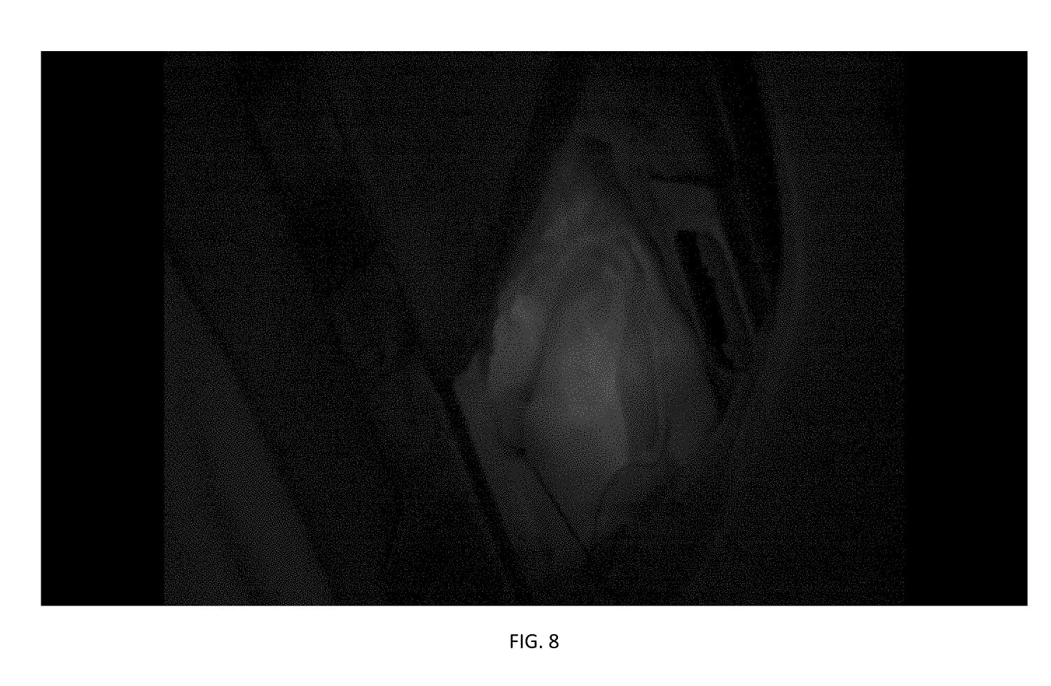

FIG. 8 is an image showing ICG dye mixed blood entering the pulmonary artery of a live swine. The aorta is also visible.

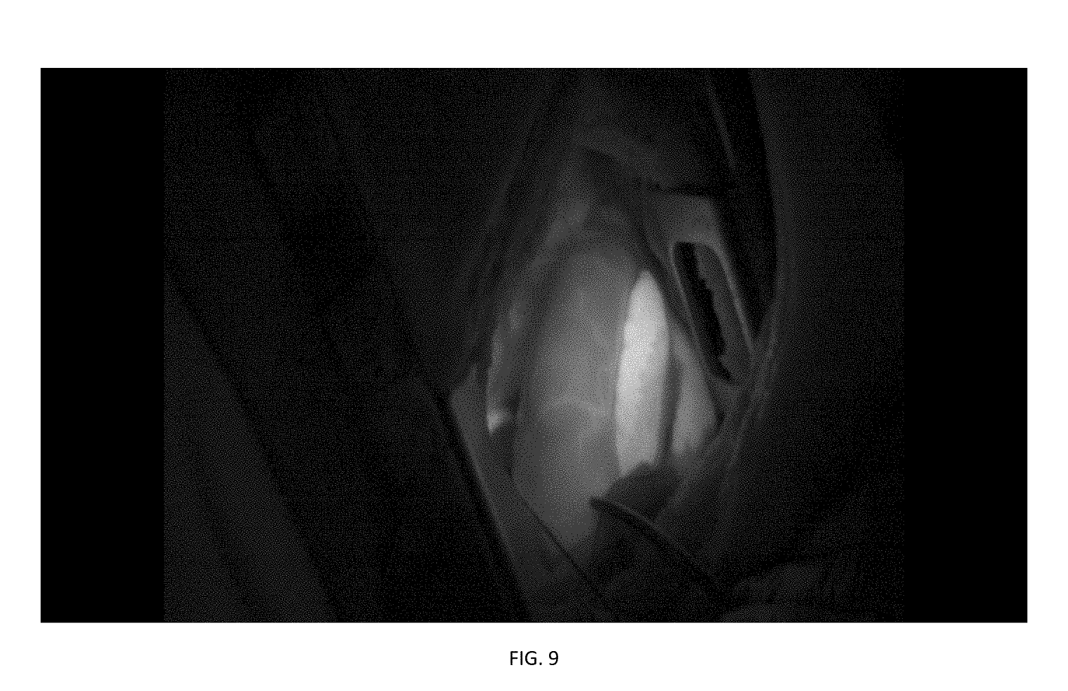

FIG. 9 is an image showing ICG dye mixed blood leaving the pulmonary artery of a live swine. The left atrial appendage is thin walled, and therefore it is visualized as much brighter.

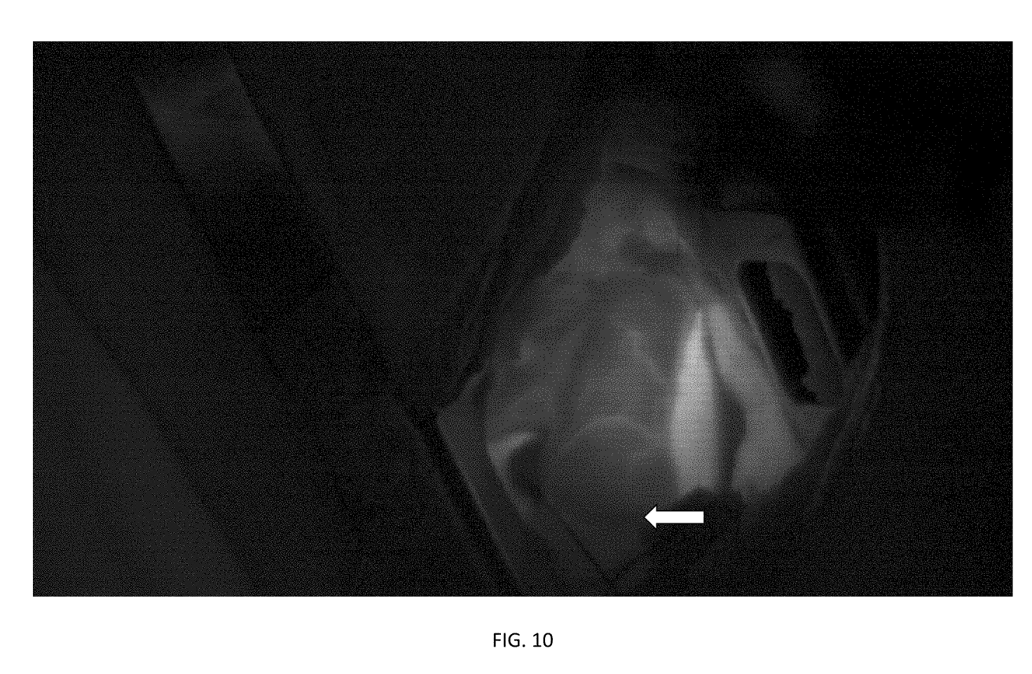

FIG. 10 is an image of a heart of a live swine. The valve can be seen faintly as a dark structure at the base of the pulmonary valve. The valve is dark because it has displaced ICG dye mixed blood.

The portion of the valve being located by fluorescent imaging is a leaflet of the valve.

The portion of the valve being located by fluorescent imaging is a leaflet of the valve and is located beneath heart muscle tissue that is about 2 mm thickness.

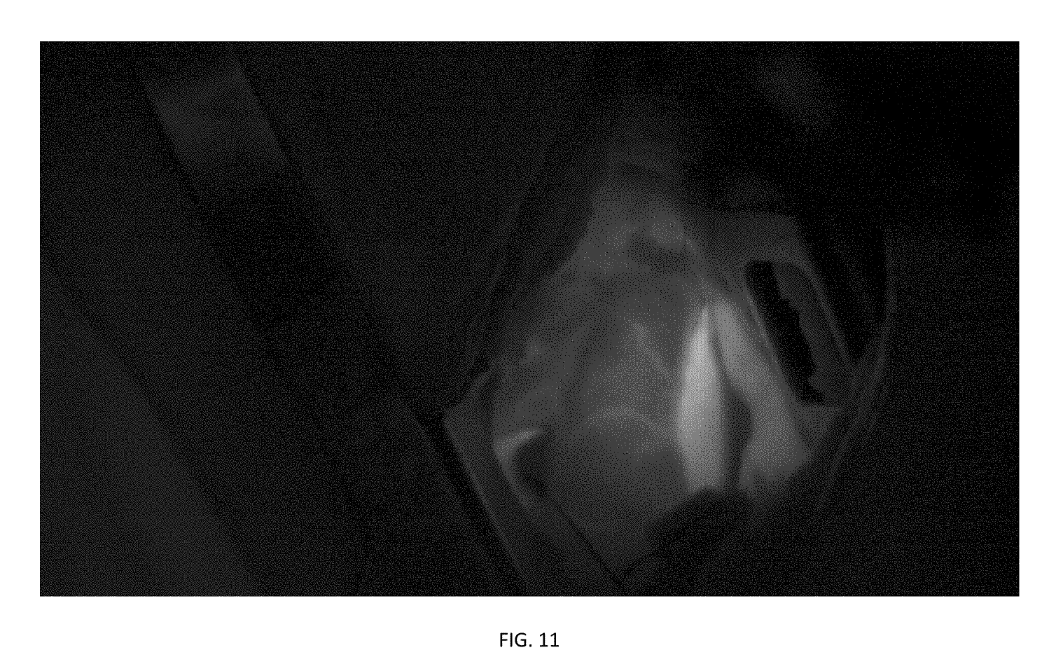

FIG. 11 is an image of a heart of a live swine, as in FIG. 10 but taken at a later point in time. The valve at the base of the artery cannot be seen in this image, thereby revealing mechanical movement of the valve has occurred.

Movement of the valve is very apparent to an individual viewing video images taken at about 30 fps.

### THIRD EXAMPLE

Immediately following the second example, the optical imaging system was used to image ICG mixed blood in intestinal tissue of live swine.

Flow of blood in gut vessels was imaged by ICG fluorescence following IV injection of 1 ml of a 1 milligram/ml ICG stock solution through the auricular catheter.

Following injection, flow of blood in the smaller blood vessels and tissues were observed. The live swine tissues were found to be fluorescent even after a period of 30 min. Images were recorded using the CMOS monochrome camera.

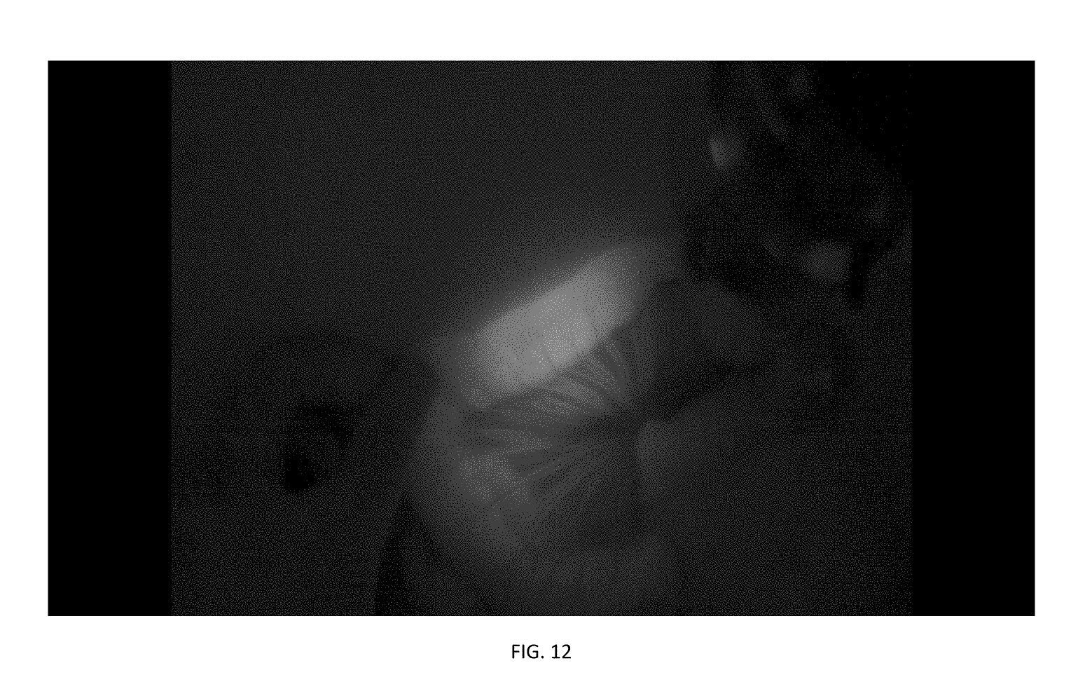

FIG. 12 is an image of a live swine that shows intestine with ICG dye mixed blood flowing through mesenteric arteries.

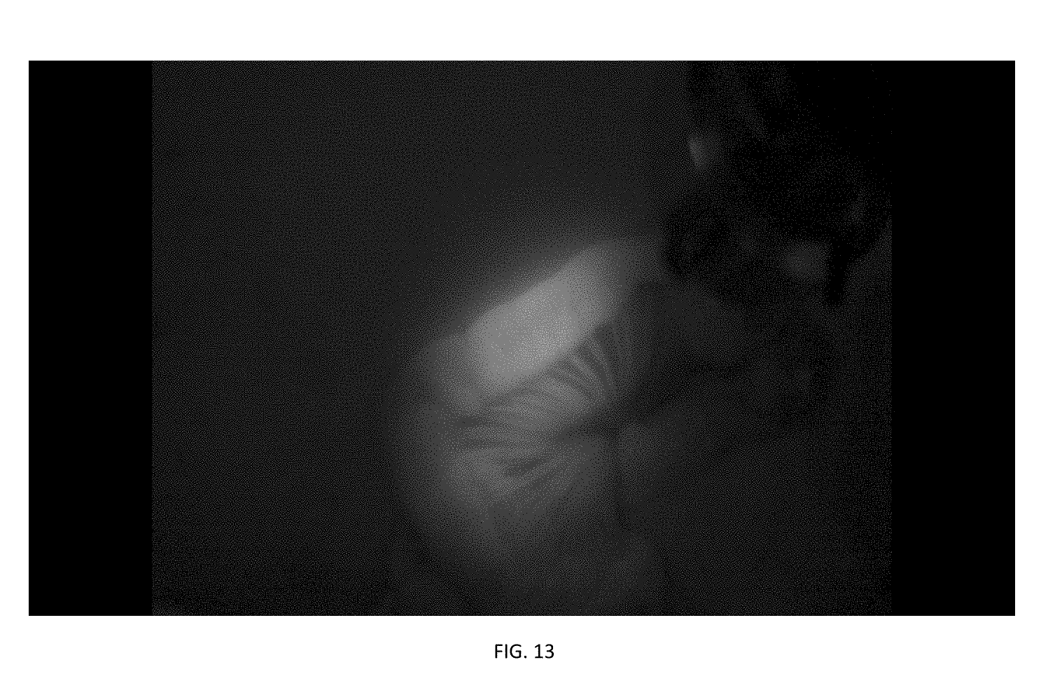

FIG. 13 is an image of a live swine with ICG dye mixed blood, taken at a later point in time. The image details an area of intestinal tissue and the mesenteric vessels.

### FOURTH EXAMPLE

The optical imaging system was used to image ICG mixed blood in live human with skin burn.

Flow of blood in live human with skin burn was imaged by ICG fluorescence following IV injection of about 5 mg of ICG in the form of a stock solution.

Following injection, flow of blood in the smaller blood vessels and tissues were observed. The live human tissues were found to be fluorescent even after a period of 30 min. Images were recorded using the CMOS monochrome camera. Two different excitation sources were employed; namely, the seven LED source used for swine, and a new twenty-three LED source. The twenty-three LED source was preferred. It provided a radiation intensity of about 4 mW/cm2.

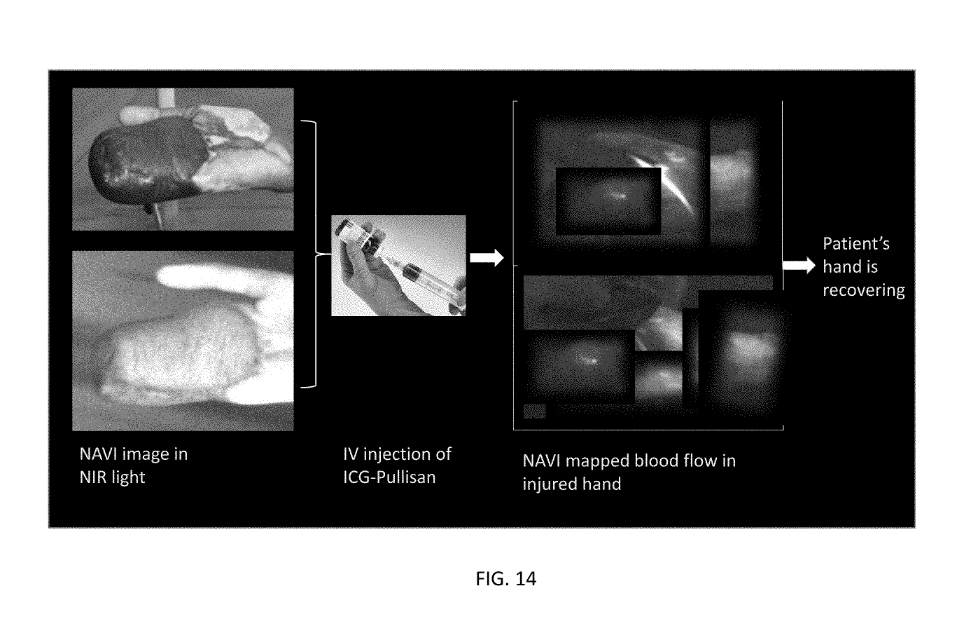

FIG. 14 is an image of a live human hand with skin burn. The two left-most photographs were taken prior to injection with ICG dye. The upper left image was taken with room ambient lighting. The lower left image was taken in NIR light. The two right-most images were taken after injection of ICG dye. The two right-most images show locations of ICG mixed blood in subsurface arteries and veins; namely, the lighter colored areas, of the live human hand with skin burn.

In view of the above, it will be seen that the several objects of the invention are achieved.

As various changes could be made in the above-described process for constructing the excitation radiation source and the optical fluorescence imaging system without departing from the scope of the invention, it is intended that all matter contained in the above description be interpreted as illustrative and not in a limiting sense.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described example embodiments, but should be defined only in accordance with the following claims appended hereto and their equivalents.

What is claimed is:

1. An optical excitation radiation source for use in an optical fluorescence imaging system, the optical excitation radiation source comprising:
an excitation source having a multiplicity of LEDs to emit radiation;
an optical reflector with a multiplicity of reflecting facets located on an interior surface to direct the radiation;
a short wavelength pass optical filter having an optical density value for radiation directed at the short wavelength pass optical filter;
a power source for the multiplicity of LEDs; and
a focusing lens.

2. The optical excitation radiation source as set forth in claim 1, wherein said multiplicity of LEDs emit in the ultraviolet, visible and infrared spectral regions.

3. The optical excitation radiation source as set forth in claim 1, wherein said multiplicity of reflecting facets are about 1 mm×1 mm in dimension, and about 1 mm high.

4. The optical excitation radiation source as set forth in claim 1, wherein said multiplicity of LEDs and said optical reflector can be replaced with a second multiplicity of LEDs and a second reflector to allow imaging with a multiplicity of fluorescent dyes without recourse to altering the mechanical structure of said optical excitation radiation source.

5. The optical excitation radiation source as set forth in claim 1, wherein a total volumetric size of said optical excitation radiation source is less than about 350 cm³.

6. The optical excitation radiation source as set forth in claim 1, wherein said power source further comprises a battery or an electronic power supply to power the multiplicity of LEDs.

7. The optical excitation radiation source as set forth in claim 1, wherein said optical excitation radiation source is portable.

8. The optical excitation radiation source as set forth in claim 1, wherein said short wavelength pass optical filter has the optical density value of at least 4.

9. An emission imaging camera system for use in an optical fluorescence imaging system, the emission imaging camera system comprising:
an imaging camera with a dynamic range and with an image output capability;
a lens for focusing incoming radiation from a fluorescent dye in a tissue;
an emission filter located at an entry to said lens with a pass band value centered near maximum emission from said fluorescent dye in said tissue, and wherein said emission filter excludes radiation from an optical excitation radiation source incident on said tissue; and
a hood attached to a distal end of said lens.

10. The emission imaging camera system as set forth in claim 9, wherein said imaging camera has the dynamic range of at least 72 dB.

11. An optical fluorescence imaging system comprising:
an optical excitation radiation source, wherein the optical excitation radiation source further comprises an excitation source having a multiplicity of LEDs to emit radiation, an optical reflector with a multiplicity of reflecting facets located on an interior surface to direct the radiation, a short wavelength pass optical filter having an optical density value for radiation directed at the short wavelength pass optical filter, a power source for the multiplicity of LEDs, and a focusing lens; and
an emission imaging camera system, wherein the emission imaging camera system further comprises an imaging camera with a dynamic range and with an image output capability, a lens for focusing incoming radiation from a fluorescent dye in a tissue, an emission filter located at an entry to said lens with a pass band value centered near maximum emission from said fluorescent dye in said tissue, and wherein said emission filter excludes radiation from an optical excitation radiation source incident on said tissue, and a hood attached to a distal end of said lens.

12. The optical fluorescence imaging system as set forth in claim 11, wherein a sensitivity of said optical fluorescence imaging system is sufficiently high to image a leaflet of a heart valve located beneath a mammal heart muscle tissue that is about 2 mm thickness.

13. The optical fluorescence imaging system as set forth in claim 12, wherein a mammal having the mammal heart muscle tissue is selected from the group consisting of a swine, a dog, a murine, or a human.

14. The optical fluorescence imaging system as set forth in claim 11, wherein use of said optical fluorescence imaging system is available in a remote location, said remote location being either a remote emergency medical location, a military field hospital location, an agricultural field location, or a remote location in need of water quality assessment.

15. A method for using an optical fluorescence imaging system, the method comprising:
anesthetizing a subject;
exposing an organ to be imaged by the optical fluorescence imaging system;
illuminating a tissue to be imaged using an excitation radiation source;
focusing an imaging camera system on the tissue to be imaged; and
obtaining a video or image of the tissue using a lowest excitation intensity setting of the imaging camera system commensurate with obtaining a high image resolution, a high contrast, and an acceptable video frame rate or image exposure time.

16. The method for using the optical fluorescence imaging system as set forth in claim 15, wherein the method further comprises:
injecting an ICG dye into a blood stream of the subject, said ICG dye being a fluorescent dye; and
waiting until said fluorescent dye enters said tissue to be imaged before obtaining said video or image of said tissue.

17. The method for using the optical fluorescence imaging system as set forth in claim 15, wherein said subject is a live animal.

18. The method for using the optical fluorescence imaging system as set forth in claim 15, wherein said subject is a human patient.

19. The method for using the optical fluorescence imaging system as set forth in claim 15, wherein said excitation radiation source,
emits radiation via a multiplicity of LEDs,
directs said radiation with an optical reflector with a multiplicity of reflecting facets located on an interior surface of the excitation radiation source, and
directs said radiation through a short wavelength pass optical filter having an optical density value onto a focusing lens.

20. The method for using the optical fluorescence imaging system as set forth in claim 15, wherein said imaging camera system,
contains a dynamic range of about 72 dB,
utilizes a lens for focusing incoming radiation from said fluorescent dye in said tissue; and
excludes radiation from said excitation radiation source incident on said tissue with an emission filter located at an entry to said lens with a pass band value centered near maximum emission from said fluorescent dye in said tissue.
